(19) United States
(12) Patent Application Publication
(10) Pub. No.: US 2024/0423467 A1
(43) Pub. Date: Dec. 26, 2024

(54) METHOD, DEVICE, AND COMPUTER PROGRAM PRODUCT FOR DETERMINING A SENSITIVITY OF AT LEAST ONE EYE OF A TEST SUBJECT

(71) Applicant: Rodenstock GmbH, Muenchen (DE)

(72) Inventors: Stephan Trumm, Muenchen (DE); Adam Muschielok, Miesbach (DE); Yohann Bénard, Muenchen (DE); Anne Seidemann, Muenchen (DE); Katrin Nicke, Neuried (DE); Nadine Jung, Muenchen (DE)

(21) Appl. No.: 18/719,591

(22) PCT Filed: Dec. 14, 2022
(86) PCT No.: PCT/EP2022/085866

§ 371 Date: Jun. 13, 2024

(30) Foreign Application Priority Data
Dec. 14, 2021 (DE) 10 2021 133 152.8

(51) Int. Cl. A61B 3/06; A61B 3/032; G02C 7/02

(52) U.S. Cl. CPC A61B 3/032; A61B 3/063; G02C 7/024

(57) ABSTRACT

A method for determining the sensitivity of at least one eye of a test subject based on at least two provided pairs of values for visual acuity and refraction, wherein at least one of the pairs of values for visual acuity and refraction. The method includes: projecting a target with an adjustable target refraction into the at least one eye of the test subject, wherein the target is configured to verify a predetermined visual acuity; and determining a visual acuity limiting refraction of the at least one eye of the test subject associated with the predetermined visual acuity by varying the target refraction of the target projected into the at least one eye of the test subject and detecting a test subject action which results in a determination that the identifiability of the target for the test subject has changed at the time of the test subject action.

### TECHNICAL FIELD

The invention relates to a method, to a device and to a corresponding computer program product for determining the sensitivity of at least one eye of a test subject or spectacle wearer. Furthermore, the invention relates to a method and to a device for calculating, optimizing or evaluating a spectacle lens for the at least one eye of the test subject taking into account the sensitivity of the at least one eye of the test subject, determined according to the invention. Furthermore, the invention relates to a method and to a device for manufacturing a spectacle lens, and to a spectacle lens manufactured using a method or a device of this kind.

### BACKGROUND

The published patent application WO 2013/104548 A1 describes optimizing a spectacle lens based on the wavefronts determined in the direct calculation of light through the eye. The wavefronts are evaluated at a plane in the eye rather than at the vertex sphere (VS), as is standard, and are therefore dependent on the properties of the eye. Using this method, the influence of the cornea and also all other individual properties of the eye, such as the deviations in the anterior chamber depth or other geometric parameters of the average population, can be included in the optimization of the spectacle lens directly by way of the wavefronts. The basis for this optimization method is a target function which, except for the calculated wavefront properties (including their higher-order aberrations (HOA)), is also dependent on target specifications and weightings, which can be required for certain properties of the wavefronts in the eye.

In the method for optimizing a spectacle lens according to the prior art, a spectacle lens is optimized by minimizing or maximizing a target function in which actual values and corresponding target values of at least one imaging property or aberration of the spectacle lens are included. The at least one imaging property or aberration can constitute a direct quantification of a wavefront deviation from a reference wavefront. An exemplary target function is e.g. the function:

$$F = {\sum }_{i} [ {{G}_{R , i} ( {R}_{act} ( i ) - {R}_{targ} ( i ) )}^{2} + {{G}_{A , i} ( {A}_{act} ( i ) - \text{?} ( i ) )}^{2} + … ] ,$$

$$\text{?} \text{indicates text missing or illegible when filed}$$

where:
i (i=1 to N) denotes an evaluation point on the spectacle lens;
R_{act}(i) denotes the actual spherical effect or the refractive error at the ith evaluation point;
R_{targ}(i) denotes the target spherical effect or the target refractive error at the ith evaluation point;
Ast_{act}(i) denotes the astigmatism or the astigmatic error at the ith evaluation point;
Ast_{targ}(i) denotes the target astigmatism or the target astigmatic error at the ith evaluation point.

The variables G_{R,i}, G_{A,i}, . . . are weights of the relevant imaging property or aberration that are used in the optimization.

The imaging properties or aberrations of the spectacle lens can be evaluated at the vertex sphere or at an evaluation plane or evaluation surface in the eye, as described in WO 2015/104548 A1.

It was later found that direct quantification of a wavefront deviation in diopters without taking into account the effective pupil size owing to the depth of focus that is dependent thereon is not the best-possible criterion for describing and assessing a spectacle wearer's perception through a spectacle lens. On the basis of this knowledge, it is proposed in DE 10 2017 007 663 A1 to take the visual acuity (sharpness of vision) into account in the target or power function directly. The visual acuity included in the target or power function is dependent on at least one imaging property or aberration of a spectacle lens system by way of an assignment, the at least one imaging property or aberration being able to be evaluated at a suitable evaluation surface (e.g. at the vertex sphere or in the eye). The spectacle lens system can consist of at least one spectacle lens (e.g. a spectacle lens of refractive spectacles). The spectacle lens system, however, preferably comprises further components such as a model eye or eye model, which can be based on average values for spectacle wearers or on at least one individual parameter of the spectacle wearer's eye. In other words, the spectacle lens system, which is based on assigning at least one imaging property or aberration to the visual acuity of the spectacle wearer, can be a spectacle lens/eye system.

As described in DE 10 2017 007 663 A1, an exemplary target or power function, which is dependent on the visual acuity V by way of assigning the at least one imaging property or aberration ΔU_{s,j} to the spectacle wearer's visual acuity or an average spectacle wearer, can e.g. have the following structure:

$$\text{?} = {\sum }_{i} [ \text{?} {( {V}_{act} ( Δ {U}_{s , j} ( i ) ) - {V}_{targ} ( Δ {U}_{s , j} ( i ) ) )}^{2} + … ] .$$

$$\text{?} \text{indicates text missing or illegible when filed}$$

In the above formula, V(ΔU_{s,j}(i)) denotes a function which describes the dependency of the visual acuity on at least one imaging property or aberration of a spectacle lens system at the ith evaluation point (i=1, 2, 3, . . . , N) at an evaluation surface. In other words, V(ΔU_{s,j}(i)) describes an exemplary assignment of at least one imaging property or aberration of a spectacle lens system to the visual acuity of the test subject or spectacle wearer or an average spectacle wearer when viewing an object through the spectacle lens system. The argument ΔU_{s,j} is generic and can denote any imaging property or aberration of a spectacle lens system which describes the effect of the spectacle lens system on a light beam emerging from an object or the difference in the effects of the spectacle lens system on a light beam emerging from an object and on a reference light beam converging on the retina of the eye. In this case, one or more imaging properties or aberrations can be included in the target or power function and can be evaluated, with the subscript j,j≥1 denoting the jth imaging property or aberration.

V_{act}(ΔU_{i,j}(i)) denotes the visual acuity, which is determined on the basis of the assignment and the actual value of the at least one imaging property of the spectacle lens to be calculated (e.g. to be optimized) or evaluated at the ith evaluation point, and V_{targ}(ΔU_{i,j}(i)) denotes the corresponding target value of the visual acuity.

The at least one imaging property or aberration can be calculated or evaluated at a suitable evaluation surface. The subscript “s” accordingly stands for any evaluation surface of the at least one imaging property or aberration ΔU_{s,j}. The evaluation surface can e.g. be a plane (evaluation plane) or a curved (e.g. spherical) surface. The evaluation surface can e.g. be the vertex sphere or a surface in the eye, e.g. one of the following planes or surfaces:
a plane or an (e.g. spherical) surface behind the cornea,
the front surface of the eye lens or a plane tangential to the front surface of the eye lens,
the rear surface of the eye lens or a plane tangential to the rear surface of the eye lens,
the plane of the exit pupil (EP), or
the plane of the rear lens surface (L2).

The variable G_{s,iso,i}^{V} denotes the weighting of the visual acuity predetermined by the assignment to the imaging property ΔU_{s,j} at the ith evaluation point.

In this case, for example one of the visual acuity models described in DE 10 2017 007663 A1 or any other suitable visual acuity model (which in particular describes the visual acuity as a function of the refraction or false refraction) can be used, and specifically can preferably be incorporated into the target function of an optimization in combination with a specification, such as the visual acuity model in conjunction with a transformation of the target specifications and weightings. It should be noted at this point that, as part of this description, a sensitivity metric (as described below) can preferably be used on the basis of a visual acuity model of this kind (as a functional dependency of a visual acuity value on the refraction/false refraction. In particular, a preferred sensitivity metric could be used as a derivation of a visual acuity model (i.e. the function of the visual acuity value of the refraction/false refraction) in accordance with the refraction/false refraction.

By means of the target function, a spectacle lens can likewise be evaluated, with the actual value of the at least one imaging property of the spectacle lens to be evaluated being calculated at at least one evaluation point of the spectacle lens to be evaluated and compared with the corresponding target value.

As is also clear from DE 10 2017 007 663 A1, first and foremost, knowledge of so-called sensitivity, i.e. the change in visual acuity with the false refraction, is useful for calculating, optimizing and/or manufacturing highly customized, high-quality spectacle lenses. For instance, assigning the at least one imaging property or aberration of a spectacle lens system to the visual acuity of the spectacle wearer, or the function V(ΔU_{s,j}(i)), can be parametrically dependent on the measured output visual acuity and/or the determined sensitivity of the spectacle wearer.

In the context of the present invention, the sensitivity is a variable (in particular a phenomenological variable) used in spectacle optics and ophthalmology or a parameter by means of which the dependency of the visual acuity on a false refraction can be described or stated. The sensitivity of an eye is understood in particular to mean the change in the visual acuity of the eye in the event of a change in a false refraction. In particular, the sensitivity can be defined as the derivation of the visual acuity in accordance with the false refraction or as the local derivation of the visual acuity in accordance with the false refraction under a certain false refraction. In this case, the false refraction is a deviation of an effect or refraction applied to the at least one eye of the test subject during the visual acuity determination from an ideal refraction determined or known for the at least one eye. The ideal refraction (also referred to as optimal refraction or target refraction in the following) can e.g. be determined from a conventional objective and/or subjective refraction measurement. In particular, the sensitivity describes how much the visual acuity changes when an optical effect or correction in front of the eye changes. The sensitivity can in particular be described quantitatively by means of a sensitivity metric and/or by means of a visual acuity model.

The sensitivity of the at least one eye of a test subject can thus be taken into account when calculating and/or producing individual spectacle lenses, in particular when producing multifocal spectacle lenses such as ophthalmic spectacle lenses. Spectacle lenses can have transitions between regions having different optical corrections, i.e., for example, transitions between a vision point for distance vision and a vision point for near vision. These transitions between spectacle lens regions having different optical corrections can be configured differently. In this case, reference is for example made to hard transitions or soft transitions, depending on how severe or gentle the change in refraction is along the transition. In highly customized, high-quality spectacle lenses, a transition of this kind (but also other regions of the spectacle lens) can in particular be adjusted to the sensitivity of the at least one eye of the test subject or spectacle wearer.

The presence of at least two applied effects and the visual acuity achieved thereby in each case is required for determining the sensitivity of the at least one eye of a test subject to a lack of focus. Relevant models and corresponding formulas for calculating the sensitivity are described below. According to the prior art, quantized effects are applied to the test subject or the at least one eye of the test subject to determine the sensitivity (e.g. in increments of 0.25 dpt using conventional trial lens sets). On the basis of a visual acuity chart having optotypes in quantized sizes and/or quantized visual acuity levels, the corresponding visual acuity is determined for the applied effects. Furthermore, the optimum correction (or the optimum refraction or target refraction) needs to be determined for the test subject in order for it to be possible to convert the applied effects into a false refraction.

In the context of the present invention, it has been found that the double quantization associated with the conventional methods results in a high level of measurement uncertainty. In addition, it has been found that the conventional method may not only be complex, but also have a negative psychological impact, since the at least one eye of the test subject is provided with a poorer correction after determining the optimum refraction and the test subject then has to complete visual tasks with this poorer correction in order to determine the sensitivity. This sequence is necessary in the conventional approach since a defined fogging for the visual acuity measurement can only be set when the optimum refraction is known.

One problem addressed by the present invention is therefore to determine the sensitivity of the at least one eye of the test subject, which is required in particular for calculating, optimizing, evaluating and/or manufacturing highly customized, high-quality spectacle lenses, in an improved manner, in particular in a simple and rapid manner. Furthermore, one problem addressed by the present invention is to provide a method and a device for calculating, optimizing, evaluating and manufacturing spectacle lenses that are highly customized and of high quality owing to taking into account the sensitivity of the at least one eye of the test subject. One problem addressed by the present invention is also to provide improved spectacle lenses of this kind. These problems are solved by the subjects of the independent claims. Advantageous embodiments are found in the dependent claims.

### SUMMARY

A first independent aspect for solving the problem relates to a method for determining the sensitivity of at least one eye of a test subject based on at least two provided pairs of values for visual acuity and refraction. In this case, at least one of the pairs of values for visual acuity and refraction is provided by the following steps:
projecting a target with an adjustable target refraction into the at least one eye of the test subject, wherein the target is configured to verify a predetermined visual acuity; and
determining a visual acuity limiting refraction of the at least one eye of the test subject associated with the predetermined visual acuity by varying the target refraction of the target projected into the at least one eye of the test subject and detecting a test subject action which results in a determination that the identifiability of the target for the test subject has changed at the time of the test subject action.

As already explained at the outset, the “sensitivity” (to a lack of focus) of at least one eye of the test subject is understood to mean the dependency of the visual acuity of the at least one eye of the test subject on a false refraction, wherein the “false refraction” is a deviation of an effect or refraction applied to the at least one eye of the test subject during the visual acuity determination from an ideal or optimum refraction (target refraction) determined or known for the at least one eye.

The “visual acuity” is a measure of the (central) sharpness of vision of the at least one eye of the test subject. Usually, the visual acuity is determined under bright light conditions. In particular, the visual acuity can be defined as the inverse value of the smallest perceivable gap in the standard test character, the Landolt ring. In humans, visual acuity can be determined by means of an eye test. To do this, the test subject is presented with optotypes, and it is apparent from the test subject's answers whether the test subject has correctly identified them. Visual acuity is dependent on which optotypes the test subject can identify at the set or applied refraction. The optotypes generally have a defined size, brightness, shape and contrast. The optotypes can be displayed on or projected onto a chart. The use of a projector instead of a chart has the advantage of being independent of the testing distance. There are DIN standards for a reproducible visual acuity test. According to these standards, the standard optotype is the so-called Landolt ring, a ring of defined width with a gap of the same width, which can be arranged in eight different directions. By identifying the direction of the gap, the test subject demonstrates that their resolving power corresponds at least to the width of the gap. In practice, however, because they are easier to understand, standardized images of numbers are used as optotypes. There are also other standardized optotypes, such as the “Snellen E”, the “Pflüger trident E”, in which the middle stroke is shorter, as well as others, which are suitable for testing the visual acuity of illiterate persons and pre-school children, as well as for non-verbal communication.

When determining visual acuity, a distinction is made between those with correctors, such as spectacles or contact lenses, and those without correctors. Here, the sharpness of vision without correctors is referred to as natural visual acuity. The abbreviations “s.c.” (“sine correctione”, Latin for “without correctors”) and “c.c.” (“cum correctione”, Latin for “with correctors”) are often also used.

The sensitivity of the at least one eye can in particular be determined on the basis of a sensitivity metric. By using a sensitivity metric, a sensitivity can be calculated even when the applied refraction values are not a predetermined distance from one another.

The sensitivity metric represents the dependency of the visual acuity on a (false) refraction. In this case, the distance between two refraction values can be part of a sensitivity metric. The sensitivity metric can be defined in the metric space of the refraction values. A visual acuity value can be assigned to each refraction value of the sensitivity metric, or vice versa. The refraction can, for example, be defined in an at least three-dimensional space. For instance, a refraction value can be described by the coordinates s, c and a. Here, s can be dependent on the strength of an optical correction of the sphere, con the strength of an optical correction for a cylinder, and a on the axis position of this cylinder. In this metric space of the refraction values, at least the refraction values for a predetermined first visual acuity and a predetermined second visual acuity can be determined, and are therefore known in the calculation of the sensitivity. The sensitivity metric can be used to determine the sensitivity on the basis of two different refraction values, which in principle are arbitrary. By using a sensitivity metric of this kind, the determination of the sensitivity is independent of visual acuity measurements with predetermined refraction values, as is standard in conventional methods. In this case, the determination of the sensitivity can be independent both of visual acuity measurements with at least one predefined and/or fixed refraction distance from the refraction result (or from the optimum refraction or target refraction) and of visual acuity measurements with at least one predetermined and/or fixed relative refraction distance between the two applied refractions. Therefore, it can be made easier, both for refractionists and test subjects, to determine the measurement data required for the sensitivity determination.

### Embodiments of a Sensitivity Metric

The sensitivity can be calculated by means of a metric space in which different refraction values represent individual points. A refraction value can e.g. be represented three-dimensionally, e.g. by the coordinates s, c and a. Here, s can be dependent on the strength of a spherical correction and e.g. be indicated in diopters (which can also be abbreviated to dpt). c can be dependent on the strength of a cylindrical correction and e.g. be indicated in diopters. a can be dependent on the axis position of the cylindrical correction and e.g. be indicated in degrees, e.g. from 0 to 180°. Alternatively, other coordinates can be used for this purpose.

In the following, by way of example it is assumed that the best refraction (in the context of this description, also referred to as optimum or ideal refraction), i.e. in particular a particular objective and/or subjective refraction result, is denoted in this sensitivity metric by s₀, c₀, and α₀ and the associated visual acuity by v₀. When carrying out the method, at least two pairs of values for visual acuity and refraction are provided. In general, n refractions sᵢ, cᵢ, αᵢ can be provided with the associated visual acuity vᵢ, where iε[1, . . . , n] and n>2. In this case, at least one pair of values for visual acuity and refraction for the at least one eye of the test subject can already be known and provided as a known pair of values. Providing in particular includes determining and/or measuring.

In a possible sensitivity metric, the distance of a refraction i from the best refraction in the central sphere dᵢ and in the cylinder aᵢ is calculated using the equation (1):

$${d}_{i} = ( {s}_{i} + \frac{1}{2} \cdot {c}_{i} ) - ( {s}_{0} + \frac{1}{2} \cdot {c}_{0} ) {a}_{i} = \sqrt{{c}_{i}^{2} + {c}_{0}^{2} - 2 \cdot {c}_{i} \cdot {c}_{0} \cdot cos ( {α}_{i} - {α}_{0} )} \qquad ( 1 )$$

### Simple Bilinear Model of a Sensitivity Metric with Knowledge of a Target Refraction

In one embodiment of a bilinear model of a sensitivity metric, for the dependency of the visual acuity, the following relationship set out in equation (2) applies to each individual measurement for a refraction i. In this case, in a simplified case it can be assumed that the test subject cannot compensate for fogging by accommodation.

$$lg {v}_{i} = {m}_{d} \cdot \vert {d}_{i} \vert + {m}_{a} \cdot {a}_{i} + lg {v}_{0} \qquad ( 2 )$$

Here, m_{d} stands for the sensitivity with a spherical distance and mₐ stands for the sensitivity with a cylindrical distance. This division between a spherical and a cylindrical false refraction can be used to take into account that test subjects can react very differently to these two components of a false refraction. For instance, it can be determined from data from D. Methling: Bestimmung von Sehhilfen [Determination of visual aids], 2^{nd} edition, Ferdinand Enke Verlag, Stuttgart 1996, that for example the equations (3) are applicable in the average population, determined empirically:

$${m}_{d} = \frac{2}{1 dpt} \cdot lg 0.5 = - 0.601 {dpt}^{- 1} {m}_{a} = \frac{1}{1 dpt} \cdot lg 0.5 = - 0.301 {dpt}^{- 1} \qquad ( 3 )$$

In general, the above equation (2) has the independent parameters mₐ, m_{d}, v₀. Therefore, the equation system (2) can be clearly solved with three measurements i=1,2,3 of refractions (s₁, c₁, α₁; s₂, c₂, α₂; s₃, c₃, α₃) at three (in particular predetermined) different visual acuity values v₁, v₂, v₃, to give the equation system (2a):

$${m}_{a} = - \frac{1}{denominator} ( log ( v \text{?} ) + log ( v \text{?} ) + log ( v \text{?} ) ) \\ {m}_{d} = - \frac{1}{denominator} ( log ( v \text{?} ) + log ( v \text{?} ) + log ( v \text{?} ) ) \\ log {v}_{0} = - \frac{1}{denominator} ( log ( v \text{?} ) + log ( v \text{?} ) + log ( v \text{?} ) ) \\ where denominator = ( {a}_{2} - {a}_{3} ) \vert {d}_{1} \vert + ( {a}_{3} - {a}_{1} ) \vert {d}_{2} \vert + ( {a}_{1} - {a}_{2} ) \vert {d}_{3} \vert \qquad ( 2 a )$$

$$\text{?} \text{indicates text missing or illegible when filed}$$

Here, a visual acuity measurement can take place under optimum correction conditions, for example, i.e. at the target refraction (in particular determined from an objective and/or subjective refraction measurement). The following then applies where i=3: (s₃, c₃, α₃)=(s₀, c₀, α₀). Then, under these optimum correction conditions, a₃=a₀=0 and d₃=d₀=0. Therefore, the third of the equations (2a) is automatically fulfilled. The other equations then take the following form of the equation system (4):

$${m}_{a} = - \frac{1}{denominator} ( \vert {d}_{2} \vert log ( {v}_{1} / {v}_{0} ) - \vert {d}_{1} \vert log ( {v}_{2} / {v}_{0} ) ) \\ {m}_{d} = - \frac{1}{denominator} ( - {a}_{2} log ( {v}_{1} / {v}_{0} ) + {a}_{1} log ( {v}_{2} / {v}_{0} ) ) \\ where denominator = {a}_{2} \vert {d}_{1} \vert - {a}_{1} \vert {d}_{2} \vert \qquad ( 4 )$$

The equation system (4) thus provides an exemplary embodiment of a simplified bilinear model of a sensitivity metric. The equation system (4) can be solved with the knowledge of the target refraction and with the knowledge of two additional refraction values for two additional visual acuity values (for i=1,2). Therefore, the sensitivity can be determined from the equation system (4). The sensitivity describes the dependency of the visual acuity on the (false) refraction. This can e.g. be described by the values mₐ and m_{d}.

If more than two additional refractions are measured at predetermined visual acuity values in addition to the visual acuity v₀ at the target refraction, the sensitivity can be determined more accurately by m_{d} and mₐ being determined from all the data by means of a compensation method, e.g. the method of least squares. Furthermore, outliers can be excluded from the measurement data in order to improve the quality of the sensitivity determination.

### Simplified Linear Model of a Sensitivity Metric with Knowledge of a Target Refraction

In a more simplified, less customized model of the sensitivity metric, e.g. when only one measurement is available with a false refraction i=1, a relationship between the spherical and the cylindrical refraction distance can be assumed according to equation (5):

$${m}_{d} = m {m}_{a} = f \cdot {m}_{d} = f \cdot m \qquad ( 5 )$$

Here, the parameter f can be derived from empirical values and can e.g. be a scalar. With the assumption according to equation (5), the equation system (2) simplifies to give the following equation (6):

$$lg {v}_{i} = m \cdot ( {d}_{i} + f \cdot {a}_{i} ) + lg {v}_{0} lg {v}_{i} = m \cdot ( {a}_{i} + f \cdot {d}_{i} ) + lg {v}_{0} \qquad ( 6 )$$

Therefore, the sensitivity m can be determined from a measurement with a false refraction i from equation (7):

$$m = \frac{{a}_{i} + f \cdot \vert {d}_{t} \vert}{lg {v}_{i} lg {v}_{0}} \qquad ( 7 )$$

A value for f can be derived from relevant technical literature, e.g. f=1/2 can be applied, derived from Applegate, R. A, Sarver, E. J, Khemsara: “Are all aberrations equal?”, J Refract Surg. 2002, 18: pages 556-562. Alternatively, f=1 can be applied, derived from Atchison et al.: “Blur limits for defocus, astigmatism and trefoil”, VisionResearch, 2009.

In this case, a linear relationship does not need to be assumed for equation (5). Alternatively, more complex relationships can be formulated and the sensitivity can be derived therefrom, e.g. on the basis of a number of the independent parameters and/or the refraction measurements, by being introduced into accordingly resolved relationships; cf. equations (4) and (7). The sensitivity can also be derived from a compensation method, e.g. the method of least squares.

### Further Models of a Sensitivity Metric with Knowledge of the Subjective Refraction

The sensitivity can also be calculated on the basis of another model. For instance, models are known from R. Blendowske, Unaided Visual Acuity and Blur: “A Simple Model”, Optometry and Vision Science, Vol. 92, No. 6, 2015, which are characterized by their particularly simple nature and by being based on only a few parameters. Simple models of this kind are particularly suitable for calculating the sensitivity and for the adjustment in a situation with low data, for example because it can effectively avoid overfitting.

If a larger number of parameters are individually available, a model having many different parameters is more suitable, as described in DE 10 2017 007 663 A1, for example.

In principle, a plurality of different models can be used. The model used in a given case can be dependent on the number of pairs of values for visual acuity and refraction that are provided or determined. When there is a sufficiently large number of pairs of values for visual acuity and refraction, relatively complex, not necessarily linear models can be formulated, the parameters of which can be adjusted to the measurements.

The models listed above by way of example can be generalized, e.g. by a function describing the sharpness of vision comprising contours of a constant sharpness of vision in the power vector space, which contours correspond to ellipsoids or ovoids containing the point of maximum sharpness of vision. This can take place similarly to a method presented in A. Rubin and W. F. Harris: “Closed Surfaces of Constant Visual Acuity in Symmetric Dioptric Power Space”, Optometry and Vision Science, Vol. 78, No. 10, 2001. In this case, axis ratios can individually differ in a range of 0.25 to 4. Instead of individually measured values, means, medians or other estimated values of the corresponding model parameters of the population can be used to calculate the sharpness of vision.

In one exemplary embodiment, a generalization of the above equation (6) results in different factors f, e.g. in equation (8):

$$lg {v}_{i} = {\{ ( {d}_{i} , {a}_{i}^{o r t} , {a}_{i}^{o b l} ) R [ \begin{matrix}{m}_{1}^{2} & 0 & 0 \\ 0 & {m}_{2}^{2} & 0 \\ 0 & 0 & {m}_{3}^{2}\end{matrix} ] {{R}^{T} ( {d}_{i} , {a}_{i}^{o r t} , {a}_{i}^{o b l} )}^{T} \}}^{1 / 2} + lg {v}_{0} \qquad ( 8 )$$

Here, aᵢ^{ort} and aᵢ^{ort} denote the astigmatism of the false refraction with orthogonal (J0) and oblique (J45) axis positions, respectively, and are defined as:

$${a}_{i}^{o r t} = - \frac{{c}_{i}}{2} cos ( 2 {α}_{i} ) + \frac{{c}_{0}}{2} cos ( 2 {α}_{0} ) and \\ {a}_{i}^{o b l} = - \frac{{c}_{i}}{2} sin ( 2 {α}_{i} ) + \frac{{c}_{0}}{2} sin ( 2 {α}_{0} ) .$$

Here, R represents a rotation matrix, which determines an orientation of an ellipsoid of constant sharpness of vision in the power vector space of the vectors (dᵢ, aᵢ^{ort}, aᵢ^{obl}). The eigenvalues m₁, m₂, m₃ denote the sensitivities to fogging in the direction of the first, second and third column vector of the rotation matrix R in the power vector space.

### Embodiments of Models of a Sensitivity Metric Without Knowledge of the Target Refraction

In some embodiments, the sensitivity can be determined without knowledge or determination of the target refraction. This can take place when an associated refraction or visual acuity limiting refraction is determined for a plurality of predetermined different visual acuity values. In this case, the best refraction or target refraction can be determined from the measurement data gathered in the process. Furthermore, an actually determined best refraction can be checked by means of a model of a sensitivity metric from the measurement data.

In this case, it can be assumed that fogging, i.e. an intentional false refraction, towards minus can be compensated for by the test subject by accommodation of the at least one eye. In this case, a point at which the visual acuity curve inflects can be selected in the linear model according to the above equations (2) and (6). In non-linear models in which there is saturation, the best refraction can be calculated directly as parameters of the equation system. For this purpose, the false refraction, i.e. the distances di and aᵢ, needs to be replaced with the difference between the best refraction and the set or applied correction in the corresponding formulas, i.e. in particular right back in equation (1).

The above-mentioned embodiments of models of a sensitivity metric are examples illustrating how the sensitivity can be determined in the context of the present invention.

The target can in particular be an actual target (or real object) or a virtual target (or virtual object). In particular, the target can be an actual object or a virtually projected object (or a projected virtual object). A target can for example be implemented by a display (e.g. having one or more lenses and/or having one or more mirrors), by a light field display and/or by a Badal optometer (which allows for constant magnification despite a change in the effect) and can be projected into the at least one eye of the test subject.

A “virtual object” or “virtual target” is in particular understood to be an optical imaging system which generates wavefronts emitted from virtual object points, such that they impinge on the at least one eye of the test subject. In this case, the wavefronts generated by the virtual target (and each corresponding to a virtual object point) and impinging on the at least one eye of the test subject have an adjustable spherical curvature and/or an adjustable cylindrical curvature component, the cylindrical curvature component preferably being adjustable both with regard to the curvature value and with regard to the axis position.

The virtual position of the virtual object (target) can preferably be changed such that, in this way, different accommodation states of the at least one eye can be stimulated. In particular, the position of the virtual object can preferably be changed between a position for stimulating a distance-vision accommodation and a position for stimulating a near-vision accommodation. In addition, the position of the virtual object can preferably be adjusted such that the at least one eye of the test subject is no longer capable of accommodating to the virtual object. In this case, the virtual object (target) can only be perceived out of focus in all directions by the test subject. This means that the ciliary muscles relax. A state of this kind is referred to as a “fogged” state.

A target is projected into the at least one eye of the test subject with an adjustable or variable target refraction (or target effect). This projection can be carried out by means of an optical system, using which the effect or refraction of the target, i.e. the target refraction, can also be adjusted and/or varied. In the context of this invention, the “target refraction” is thus understood to be the refraction (applied or caused by the optical system) (in particular spherical and/or astigmatic refraction) at which the target is projected into the at least one eye of the test subject or at which the target is provided to the at least one eye of the test subject.

An optical projection into or onto the eye of the test subject is in particular considered to be the target, such that this projection generates an image on the retina of the eye which corresponds to the image of an actual object at a certain distance from the eye. This certain distance is also referred to as the virtual position for the virtual target here. In other words, within the meaning of this description, a target is in particular imaging of an object into the at least one eye of the test subject. A backlit diapositive can be used as the object, for example. Since, in the case of a virtual target, the target is not (directly) an actual object in the virtual position, by suitably constructing the optical system for the projection a virtual position can also be simulated indefinitely. This then corresponds to wavefronts that converge towards the eye (i.e. in the propagation direction).

The projection of a target (in particular a virtual target) into the at least one eye of the test subject by means of an optical system is known in principle, and therefore this will not be discussed in greater detail in the context of the present invention. For example, the projection of a target into the at least one eye of the test subject is described in K. Nicke and S. Trumm: “Brillengläser der Zukunft—Schritt 3 Der DNEye Scanner” [Spectacle lenses of the future—step 3 of the DNEye Scanner], Der Augenoptiker [The Optician], June 2012, or also in DE 10 2013 000 295 A1.

The target projected into the at least one eye of the test subject is configured to verify a predetermined, in particular predefined and/or known, visual acuity (or a predefined visual acuity level). Here, “verify a predetermined visual acuity” can in particular be understood to mean that, by means of the target, it can be determined or established (in particular on the basis of a test subject action) whether the at least one eye of the test subject reaches the predetermined visual acuity or the predetermined visual acuity level. In other words, the target specifies a certain visual acuity or a certain visual acuity level, and it can be determined whether or not the at least one eye of the test subject reaches it (in particular on the basis of the test subject action). In particular, the target is provided (in particular dimensioned) such that a predetermined visual acuity or a predetermined visual acuity level is or can be assigned to the virtual target. In other words, the target is a target having a predetermined visual acuity or a predetermined visual acuity level. This means that the test subject, in particular with an ideal refraction or with a correction of a possible ametropia of the at least one eye of the test subject, recognizes or can identify the target, provided that the at least one eye of the test subject at least reaches or has the visual acuity predetermined by the target or the visual acuity level predetermined by the target.

In particular, the target can comprise or be an optotype suitable for determining visual acuity. In this case, the dimension or size of the optotype is dependent on the predetermined visual acuity or the predetermined visual acuity level. In particular, a dimension or size of the optotype is selected such that only a test subject having a visual acuity corresponding at least to the predetermined visual acuity or the predetermined visual acuity level can recognize and/or identify the optotype.

The target can also be an image or photograph containing two or more details, the recognition of each of which can be assigned to a predetermined visual acuity or a predetermined visual acuity level. The image can in particular show objects (such as a road stretching off into the distance, a sky, a far-off hot-air balloon, etc.) which can give the observer a sense of expanse or distance. The above-mentioned details contained in the image (such as symbols or panels on a hot-air balloon or the basket of a hot-air balloon, clouds or symbols in clouds, lines on a road, symbols on signs at the side of the road, etc.) are expressly included by the term “optotype” in the context of this description. A particularly suitable symbol as the optotype is e.g. one or more concentric rings, which merge together to form a circle at a given lack of focus.

The visual acuity or visual acuity levels of a target, targets or optotype can e.g. be determined, in a known manner, by calculating the visual angle of details or by identifying test subjects who have known visual acuity properties.

After projecting the target into the at least one eye of the test subject, a visual acuity limiting refraction of the at least one eye of the test subject associated with the predetermined visual acuity or the predetermined visual acuity level is determined.

In the context of this invention, “visual acuity limiting refraction” or “visual acuity level limiting refraction” is understood to be the refraction or limiting refraction at or from which the identifiability of the target for the test subject changes. In particular, “visual acuity limiting refraction” or “visual acuity level limiting refraction” is understood to be the refraction or limiting refraction at which the test subject
a) can first recognize and/or identify the target applied to them or the virtual target that is projected into their at least one eye and is characterized by a predetermined visual acuity or a predetermined visual acuity level starting from a fogged state by varying the target refraction (applied or caused by the optical system) or
b) can no longer recognize and/or identify said target or virtual target starting from an unfogged state by varying the target refraction (applied or caused by the optical system).

The visual acuity limiting refraction is determined by varying the target refraction of the target projected into the at least one eye of the test subject and by detecting a test subject action (e.g. a message or input from the test subject, in particular an actuation of a button or joystick). The target refraction can be varied incrementally or preferably continuously. The target refraction is preferably varied monotonically and/or constantly. The test subject action signals or determines that the identifiability of the target for the test subject has changed at the time of the test subject action. In other words, by means of the test subject action the test subject signals that, at the target refraction provided or applied at the time of the test subject action, they can either recognize or identify the target for the first time or can no longer recognize or identify the target for the first time. In particular, the visual acuity limiting refraction corresponds to the target refraction or target effect provided at the time of the test subject action or applied by the optical system.

The sensitivity of the at least one eye of the test subject is determined taking into account the predetermined visual acuity or the predetermined visual acuity level and the determined associated visual acuity limiting refraction.

The method according to the invention can in particular be carried out as part of auto-refractometric or aberrometric measurements. For this purpose, at least one pair of the visual acuity level and the associated applied effect is detected. This takes place by means of a signal from the test subject during the change in the applied effect at a target having a defined visual acuity level (i.e. defined size of an optotype).

As already mentioned, at least two pairs of the visual acuity level and the associated applied effect are required for determining the sensitivity. In conventional methods, with defined applied effects it is determined which visual acuity level is reached by the test subject with each of these effects (i.e. from which size the test subject can still recognize optotypes). In the method according to the invention, however, for at least one of these pairs, the size of the optotype (and therefore the visual acuity level) remains constant and the applied effect is changed. The test subject signals when they can still recognize or can no longer recognize an optotype having a defined size.

In the present invention, by contrast with the prior art, for determining the sensitivity, the visual acuity level for a certain applied effect is not required (with a false refraction that is known or unknown a priori), but the applied effect necessary for reaching a predetermined visual acuity is required.

The approach according to the invention allows the sensitivity to be determined in a simple and rapid manner. In particular, the approach according to the invention allows the sensitivity (as a subjective measured variable) to be determined in a simple manner without great additional complexity during a normal objective refraction measurement. In particular, complex measurements can be avoided during a subjective refraction and the step in which, after determining the best refraction, the test subject is provided with a poorer correction and is supposed to complete visual tasks therewith, which has a negative psychological impact, can be omitted. In addition, the approach according to the invention can advantageously be very well linked to further measurements for determining individual parameters for advanced spectacle lenses (e.g. near-vision measurements, pupillometry, keratopigmentation) and for optometric or ophthalmological screening or to measurements for establishing findings (such as keratopigmentation, opacity, pachymetry, tomography, tonometry, or retinal imaging).

In a preferred embodiment, prior to the step of projecting a target, which is configured to verify (or determine) a predetermined visual acuity, into the at least one eye of the test subject, an objective and/or subjective refraction result (in particular a combined refraction result which is based on an objective and subjective measurement and in which other data such as lower-order and/or higher-order aberrations from the aberrometry or other biometric data such as the shape of the cornea, lens-to-retina distance, anterior chamber depth, etc.) of the at least one eye of the test subject are also determined. In the context of the invention, a “refraction result” is in particular understood to be a determined refraction value. In this way, by contrast with the previous approach, the determination of the sensitivity can be linked to one or more aberrometric or auto-refractometric measurements. In particular, the determination of the visual acuity can be linked to the measurement of auto-refractometric or aberrometric data in the non-accommodated and accommodated state.

The objective refraction value or the objective refraction result is preferably determined in a fogged state. To do this, a target (e.g. an image or photograph) can be presented to the test subject or a corresponding virtual target can be projected into the at least one eye of the test subject (by means of the optical system), which has an effect which results in the test subject only being able to recognize the target out of focus (or not completely in focus), meaning that the ciliary muscles of the at least one eye of the test subject relax. Fogging of this kind can e.g. be carried out with an additional effect, in comparison with the optimum refraction of the at least one eye of the test subject, of approx. 1.25 dpt to 1.5 dpt.

In a specific embodiment, the accommodation state of the eye can also be tracked, in order to thus obtain even more reliable values for the sensitivity.

Preferably, prior to the step of varying the target refraction, the target is projected into the at least one eye of the test subject with such a starting target refraction that the test subject can only recognize the target out of focus (or not completely in focus) and/or cannot identify it. In other words, a starting target refraction is preferably selected such that the test subject cannot bring the target or optotype into focus by accommodation. This is in particular achieved in that the starting target refraction is shifted in the plus direction in comparison with the optimum refraction of the at least one eye of the test subject. Only by changing the target refraction in the minus direction can a state be reached in which the test subject can recognize and/or identify the target or optotype. This has the additional advantage that the test subject does not know the target or optotype at first and therefore performs the test subject action at the right time with higher probability, namely only when they can actually identify the target or optotype. If, however, the test subject knows the target or optotype in advance or at the start of the measurement (owing to a corresponding starting target refraction at which they can see the target or optotype in focus), it has been found in the context of the present invention that although an approach of this kind is possible as an alternative, it is inferior to the above-mentioned preferred embodiment in terms of the accuracy and reliability of the method. This is because a test subject who already knows the target or optotype in advance often tends to signal the time at or from which they can no longer recognize and/or identify the target or optotype after the target refraction is varied in the plus direction slightly too late.

In another preferred embodiment, either prior to or following the steps of projecting a target, which is configured to verify a predetermined visual acuity, into the at least one eye of the test subject and determining a visual acuity limiting refraction associated with the predetermined visual acuity of the target, the method comprises determining an optimum refraction (target refraction) of the at least one eye of the test subject. In particular, the method can comprise determining an objective and/or subjective refraction or an objective and/or subjective refraction result. Determining an optimum refraction can also comprise determining a combined refraction or a combined refraction result on the basis of an objective and/or subjective refraction measurement, in which, in particular, other data such as lower-order and/or higher-order aberrations from the aberrometry or other biometric data such as the shape of the cornea, lens-to-retina distance, anterior chamber depth, etc., of the at least one eye of the test subject are also taken into account. In this sense, the terms “refraction” and “target refraction” (or “refraction result”) in conjunction with the “optimum refraction” are not intended to be limited to corrections of lower-order aberrations (e.g. sphere and astigmatism), but they can also include higher-order aberrations. Therefore, the term “refraction” could also generally be understood to mean “correction”. Preferably, the optimum refraction of the at least one eye of the test subject is determined in a fogged state, which can be achieved by providing a corresponding target or projecting a corresponding target into the at least one eye of the test subject (see above). Furthermore, according to this preferred embodiment, the visual acuity achieved by the at least one eye of the test subject when compensating for a possible ametropia of the at least one eye of the test subject (e.g. based on a determined optimum refraction) is determined. In other words, the visual acuity is determined once the ametropia determined by the refraction measurement has been substantially corrected, i.e. visual acuity cum correctione (VAcc), by means of an optical system or by means of lenses, the effect of which corresponds to the determined refraction result. The visual acuity can be determined using known methods. In particular, the determined optimum refraction and the measured associated visual acuity represents one of the at least two provided pairs of values for visual acuity and refraction which are used or taken into account when determining the sensitivity. In this way, it is possible to combine the determination of the sensitivity with measurements of the objective and/or subjective refraction or to integrate it in measurements of this kind. The sensitivity can therefore be determined in a rapid and simple manner, in particular in conjunction with other measurements.

In another preferred embodiment, preferably after projecting a target, which is configured to verify a predetermined visual acuity, into the at least one eye of the test subject and after determining a visual acuity limiting refraction associated with the predetermined visual acuity of the target, the method further comprises the steps of:
determining a subjective refraction result or a subjective refraction for at least one eye of the test subject;
determining the visual acuity achieved by the at least one eye of the test subject when compensating for a possible ametropia of the at least one eye of the test subject based on the determined subjective refraction result.

The determined subjective refraction and the visual acuity of the at least one eye of the test subject determined at this determined subjective refraction preferably represent one of the pairs of values for visual acuity and refraction provided by the method (or a further pair, in particular a second, third, fourth pair, etc.) for determining the sensitivity.

Furthermore, the method preferably comprises determining an optimal refraction of at least one eye of the test subject based on the subjective refraction result and an objective refraction result. The optimum refraction is in particular a combined refraction made up of the subjective and objective refraction result. Determining a combined refraction result from an objective and subjective refraction measurement is known in principle and will therefore not be explained in greater detail in the context of the present description. For example, a combined refraction can be determined in that an objective refraction measurement is first carried out and the objective refraction result is adjusted by means of a subjective refraction that is carried out subsequently. In particular, it is also possible to determine a combined refraction by forming a mean of the objective and subjective refraction.

In another preferred embodiment, the sensitivity is determined based on at least one calculated false refraction, wherein the at least one calculated false refraction is calculated based on a determined optimum refraction. In this case, the optimum refraction can be a determined objective and/or subjective refraction. The optimum refraction can in particular represent a combined refraction made up of an objective and subjective refraction.

The false refraction is preferably determined “ex post”, i.e. only after projecting a target, which is configured to verify a predetermined visual acuity, into the at least one eye of the test subject and after determining a visual acuity limiting refraction associated with the predetermined visual acuity of the target. The false refraction is preferably determined only after determining at least one pair of values for visual acuity and refraction. The false refraction is preferably determined after carrying out an objective and/or subjective refraction measurement, and in particular after determining an ideal refraction or an ideal refraction result from an objective and subjective refraction measurement. For example, in a preferred embodiment, the following steps can be performed, in particular in the stated sequence:
1) carrying out an objective refraction measurement (as part of the approach according to the invention);
2) determining at least one pair of values for visual acuity and refraction (as part of the approach according to the invention);
3) carrying out a subjective refraction measurement;
4) determining an ideal refraction or an ideal refraction result from the objective and subjective refraction measurement; and
5) calculating the false refractions and the sensitivity starting from the result from step 4, i.e. based on the determined ideal refraction or the determined ideal refraction result.

In another preferred embodiment, varying the target refraction comprises monotonically decreasing the target refraction and/or monotonically increasing the target refraction.

In another preferred embodiment, the determination of a visual acuity limiting refraction of the at least one eye of the test subject associated with the predetermined visual acuity is carried out by decreasing the target refraction and detecting a test subject action while decreasing of the target refraction, and/or by increasing the target refraction and detecting a test subject action while increasing of the target refraction, wherein each test subject action results in a determination that the identifiability of the target for the test subject has changed at the time of the respective test subject action. In this way, the “out-of-focus point” is approached from different directions. In other words, one out-of-focus point can be determined when increasing the target refraction and another out-of-focus point can be determined when decreasing the target refraction. These out-of-focus points can be different from one another and can be subsequently averaged. In particular, the sensitivity can be determined from both out-of-focus points as part of a method of least squares by means of known metrics.

In another preferred embodiment, at least two of the provided pairs of values for visual acuity and refraction are provided by the following steps:
projecting a first target with a first adjustable and/or variable target refraction into the at least one eye of the test subject, wherein the first target is configured to verify a predetermined (predefined and/or known) first visual acuity (or a predetermined first visual acuity level);
determining a first visual acuity limiting refraction of the at least one eye of the test subject associated with the predetermined first visual acuity (or the predetermined first visual acuity level) by varying (in particular continuously, monotonically and/or constantly varying) the first target refraction of the first target projected into the at least one eye of the test subject and detecting a first test subject action which results in a signal or determination that the identifiability of the first target for the test subject has changed at the time of the first test subject action;
projecting a second target with a second adjustable and/or variable target refraction into the at least one eye of the test subject, wherein the second target is configured to verify a predetermined (predefined and/or known) second visual acuity (or a predetermined second visual acuity level) which differs from the predetermined first visual acuity (or the predetermined first visual acuity level);
determining a second visual acuity limiting refraction of the at least one eye of the test subject associated with the predetermined second visual acuity (or the predetermined second visual acuity level) by varying (in particular continuously, monotonically and/or constantly varying) the second target refraction of the second target projected into the at least one eye of the test subject and detecting a second test subject action which results in a signal or determination that the identifiability of the second target for the test subject has changed at the time of the second test subject action.

In particular, the sensitivity of the at least one eye of the test subject is determined using or taking into account the predetermined first visual acuity and the determined associated first visual acuity limiting refraction, and further using or taking into account the predetermined second visual acuity and the determined associated second visual acuity limiting refraction. Preferably, the first predetermined visual acuity or the first predetermined visual acuity level of the first target is less than the second predetermined visual acuity or the second predetermined visual acuity level of the second target. For example, the first predetermined visual acuity or the first predetermined visual acuity level can have the value 0.8 log Mar, while the second predetermined visual acuity or the second predetermined visual acuity level can have the value 1.0 log Mar. Alternatively, for example, the first predetermined visual acuity or the first predetermined visual acuity level can have the value 0.4 log Mar, while the second predetermined visual acuity or the second predetermined visual acuity level can have the value 0.8 log Mar or 1.0 log Mar. It goes without saying that other values can also be selected. The change in the predetermined visual acuity or the predetermined visual acuity level from one virtual target to the next target is preferably in the range of 0.2 log Mar to 0.7 log Mar, preferably in the range of 0.2 log Mar to 0.5 log Mar, and particularly preferably in the range of 0.2 log Mar to 0.3 log Mar.

In another preferred embodiment, determining a visual acuity limiting refraction comprises measuring and/or monitoring an accommodation state of the at least one eye of the test subject, wherein measuring the accommodation state is performed in particular at least at the time of or immediately after the test subject action. The results of a measurement or monitoring of this kind can be used for controlling the progress (e.g. terminating or repeating individual steps in the event of undesired accommodation (e.g. values falling below a certain threshold)). The measurement can be carried out both continuously and only during or immediately after the test subject action. Furthermore, a measured accommodation state (sphere, cylinder, lower-order or higher-order aberrations), ideally measured at the time of the test subject action, can be included in the calculation of the sensitivity or the false refraction. For example, the accommodation value can be subtracted from the value of the distance of the applied effect from the refraction value for distance vision. Expressed as formulas, the following applies in the simplest case: The sensitivity represents the visual acuity V as a function f of the false refraction F, i.e. V=f(F). Here, the false refraction F is
without accommodation, the difference between the actually applied effect T and the ideal effect I, i.e. F=T−I; and
with accommodation, the difference between the actually applied effect T and the currently measured effect Gₐ, i.e. F=T−Gₐ.

When there is a deviation D between the ideal effect I and the measured effect when the eye is relaxed (effect G₀), the following applies: I=G₀+D. Accordingly, in this situation, F=T−(G₀+D) and F=T−(Gₐ+D), respectively. For sphere values, this formula can be used as described. For cylinder values, the cross cylinder formula should accordingly be used. Zernike coefficients (also for higher-order aberrations) or power vectors can be used analogously.

Alternatively, or additionally, determining a visual acuity limiting refraction comprises measuring and/or monitoring a pupil size (e.g. pupil radius) of the at least one eye of the test subject, wherein measuring the pupil size is performed in particular at least at the time of or immediately after the test subject action. The pupil size can e.g. be measured by means of a camera which is part of an auto-refractometer or aberrometer or by means of a separate camera. The pupil size measured at the time of the test subject action (i.e. at the out-of-focus point) or accordingly just before or after (e.g. up to 2 seconds before the out-of-focus point is reached) can be used when determining the sensitivity of the at least one eye of the test subject to a lack of focus. In particular, the measured pupil size can be used to preferably quantify the lack of focus of the image on the retina by means of a suitably parameterized eye model and known additional fogging. Instead of a complete eye model, a simpler description can also be used. For instance, the angle can be calculated at which the disk of confusion of an out-of-focus point can be observed in a given pupil and with given additional fogging (see e.g. WO 2019 034525 A1). The sensitivity can be determined as part of a visual acuity model of this kind as a deterioration in the sharpness of vision per angle of the disk of confusion.

In another preferred embodiment, the test subject is presented with a visual task with at least two, preferably at least three, particularly preferably at least four, in particular four or eight, possible different answers in order to determine a visual acuity limiting refraction, wherein the test subject can answer the visual task by means of the test subject action. Here, a “visual task” is in particular understood to mean a task that has a predetermined and thus verifiable solution. In particular, the visual task is thus a verifiable task (i.e. a visual task of which the solution is known and therefore is verifiable). In other words, the test subject action goes beyond merely communicating the recognizability or identifiability of the target. The visual task is preferably based on a “forced choice”, i.e. the test subject is “forced” to make a choice from a plurality of, or at least two or a number of, possible answers, wherein the correct answer is preferably predetermined or known. In the context of the invention, a visual task of this kind is referred to as a “forced choice” visual task. The visual task can be solved or a choice can be made e.g. by means of a joystick, using which the test subject can activate different directions. For example, the visual task can consist in the test subject having to identify the position or direction of the gap in an optotype by means of a joystick. If the optotype is a Landolt ring, for example, there are eight possible positions, and therefore eight possible answers for the test subject. It goes without saying that, in principle, other optotypes can also be used, and therefore the test subject has e.g. two, three, four, five, six, seven, etc., possible answers. This makes the method more accurate and reliable than if the test subject only has to give an unverified response (e.g. “yes” or “no”, or “recognizable” or “not recognizable”).

In another preferred embodiment, prior to the step of determining a visual acuity limiting refraction, first aberrometric data of the at least one eye of the test subject, preferably for a distance accommodation state and/or a fogged state of the at least one eye of the test subject, and in particular at a first brightness, are acquired. Furthermore, the method preferably comprises acquiring second aberrometric data of the at least one eye of the test subject for a near accommodation state of the at least one eye of the test subject, in particular at a second brightness, the value of which is below that of the first brightness. Here, the acquiring of second aberrometric data preferably occurs prior to the step of determining a visual acuity limiting refraction. In the context of this description, “aberrometric data” (or “aberrometric measurements”) are understood to mean data for describing the aberrations of an eye (measurements for obtaining these data), the information content of which corresponds at least to the term of the “defocus” order when represented with Zernike coefficients, but ideally includes higher orders (e.g. coma and spherical aberrations). In particular, the “aberrometric data” can also include or be (purely) auto-refractometric data. In particular, the acquiring of aberrometric data also includes acquiring of (purely) auto-refractometric data (i.e. sphere and/or cylinder and/or axis). A brightness in the mesopic vision regime is preferably provided as the first and second brightness (preferred light density in the range of approximately 0.003 cd/m² to approximately 30 cd/m², particularly preferably in the range of approximately 0.003 cd/m² to approximately 3 cd/m², more preferably in the range of approximately 0.003 cd/m² to approximately 0.3 cd/m², more preferably in the range of approximately 0.003 cd/m² to approximately 0.03 cd/m²). The brightness is in particular always understood to be the brightness at the location of the eye or the brightness to be detected by the eye.

Together with the acquiring of first aberrometric data and/or the acquiring of second aberrometric data (i.e. in particular at the first and/or second brightness and in the first and/or second accommodation state), first and/or second pupillometric data can further be acquired for the at least one eye of the test subject. Here, the term “pupillometric data” (or pupillometric measurements) refers to information regarding the size of the pupil (or measurements for obtaining these data) that includes at least one size indication (for example in the form of a radius) but can also reproduce the shape of the pupil in a more complex form. In addition, the pupillometric data can contain information regarding the position of the pupil (for example relative to the corneal vertex or to the optical axis of the eye).

A further aspect for solving the problem relates to a method for calculating, optimizing or evaluating a spectacle lens for at least one eye of a test subject or spectacle wearer taking into account the sensitivity of the at least one eye of the test subject, wherein the sensitivity of the at least one eye of the test subject is determined by the method according to the invention described herein.

In particular, the method for calculating, optimizing or evaluating a spectacle lens for at least one eye of a test subject comprises the following steps:
a) providing an assignment of at least one imaging property or aberration of a spectacle lens system to the visual acuity of the spectacle wearer or an average spectacle wearer when viewing an object through the spectacle lens system;
b) determining or specifying a target function for the spectacle lens to be calculated or evaluated in which the assignment from step (a) is to be evaluated;
c) calculating or evaluating the spectacle lens to be calculated or evaluated by evaluating the target function, wherein the target function is evaluated at least once.

Assigning the at least one imaging property or aberration of a spectacle lens system to the visual acuity of the spectacle wearer can be parametrically dependent on the measured output visual acuity and/or the measured sensitivity of the spectacle wearer. The calculation and/or optimization of the spectacle lens can in particular include minimizing or maximizing the target function. The method for calculating, optimizing or evaluating a spectacle lens can further include calculating at least one light beam emerging from the object for at least one viewing direction by means of wavefront calculation, beam calculation or wave-field calculation through the spectacle lens system and/or through the spectacle lens to be calculated or evaluated as far as an evaluation surface in the spectacle lens system. Furthermore, the method for calculating, optimizing or evaluating a spectacle lens can include calculating the difference, at the evaluation surface, in the light beam emerging from the object in comparison with a reference light beam converging on the retina of a model eye and determining the at least one imaging property or aberration based on the calculated difference. At least one light beam emerging from the object is preferably calculated by means of wavefront calculation, wherein calculating the difference at the evaluation surface includes calculating the wavefront difference between the wavefront of the light beam emerging from the object and the wavefront of the reference light beam converging on the retina, wherein the wavefront difference is calculated at the evaluation surface. Furthermore, the method for calculating, optimizing or evaluating a spectacle lens can include assigning a geometric-optical angle and/or a quadratic form in the geometric-optical angle space to the calculated wavefront difference, wherein the at least one imaging property or aberration is dependent on at least one component of the geometric-optical angle and/or the quadratic form.

Alternatively, or additionally, the method for calculating, optimizing or evaluating a spectacle lens can comprise the following steps:
specifying a first surface and a second surface for the spectacle lens to be calculated or optimized;
determining the course of a main beam through at least one vision point of at least one surface of the spectacle lens to be calculated or optimized into a model eye;
evaluating an aberration of a wavefront resulting along the main beam from a spherical wavefront impinging on the first surface of the spectacle lens at an evaluation surface in comparison with a wavefront converging in one point on the retina of the eye model;
iteratively varying the at least one surface of the spectacle lens to be calculated or optimized until the evaluated aberration corresponds to a predetermined target aberration.

Another aspect for solving the problem relates to a method of manufacturing a spectacle lens, comprising:
calculating or optimizing a spectacle lens according to the method according to the invention for calculating or optimizing a spectacle lens; and
manufacturing the spectacle lens calculated or optimized in this way.

In addition, the invention provides a computer program product, in particular in the form of a storage medium or a data flow, which contains a program code, which is configured to, when loaded and executed on a computer, carry out a method according to the invention, in particular for determining the sensitivity of at least one eye of a test subject and/or for calculating, optimizing or evaluating a spectacle lens and/or for manufacturing a spectacle lens. In other words, the invention provides a computer program product which comprises machine-readable program code which, when loaded on a computer, is suitable for performing the above-described method according to the invention. In particular, a computer program product is understood to be a program stored on a data medium. In particular, the program code is stored on a data medium. In other words, the computer program product comprises computer-readable instructions which, when loaded into a memory of a computer and executed by the computer, cause the computer to carry out a method according to the invention.

In particular, the invention provides a computer program product which contains a program code, which is designed and configured to, when loaded and executed on a computer, carry out a method according to the invention for determining the sensitivity of at least one eye of a test subject and/or a method according to the invention for calculating, optimizing or evaluating a spectacle lens and/or a method according to the invention for manufacturing a spectacle lens.

A further independent aspect for solving the problem relates to a device for determining the sensitivity of at least one eye of a test subject, comprising:
a target providing device for providing a target which is configured to verify a predetermined visual acuity;
an optical system for projecting the target with a target refraction into the at least one eye of the test subject, wherein the optical system is configured to adjust and vary the target refraction;
a feedback unit for detecting a test subject action in order to determine that the identifiability of the target for the test subject has changed at the time of the test subject action, in particular as a result of varying the target refraction of the target projected into the at least one eye of the test subject with the aid of the optical system; and
a visual acuity limiting refraction determination unit for detecting a visual acuity limiting refraction of the at least one eye of the test subject associated with the predetermined visual acuity, wherein the visual acuity limiting refraction determination unit is configured to detect (in particular determine and store) the target refraction caused by the optical system at the time of the test subject action.

The target providing device can e.g. comprise an electronic display or a digital screen. In particular, the display can be configured such that individual pixels of the display, different regions or different components of the display can be actuated individually, in particular to display compiled optotypes. For example, sub-segments of a ring can be displayed, using which Landolt C optotypes having differently oriented openings can be generated or displayed. Alternatively, or additionally, complete optotypes, such as letters or numbers, can also be formed as whole and in particular switchable LCD elements.

The target providing device can e.g. comprise a flap mechanism, a shifting mechanism or a rotation mechanism, which is magnetic or motorized, for example, using which different targets or images can be shown and/or replaced. The targets and images can also be partially transparent and only contain regions which are supposed to be displayed in addition to another image.

Transparent, back-lit images can also be configured such that certain parts of the image are only visible when one or more particular light sources (e.g. otherwise shaded regions or having specific wavelengths) are switched on or off.

The optical system is in particular arranged between the at least one eye of the test subject and the target providing device or the provided target. The optical system is configured to apply or bring about different target effects and to thus influence the recognizability of the target for the at least one eye of the test subject. In the simplest case, the optical system is configured to apply various spherical effects. This can e.g. be done by arranging one or more spherical lenses, for example in the form of a Badal system. Alternatively, or additionally, one or more adaptive lenses, optionally in combination with conventional lenses, can be used or arranged. In more complex cases, the optical system can be configured to apply or bring about various cylinder effects or higher-order effects in addition to or instead of spherical effects.

The optical system can comprise at least one lens having a spherical effect and/or at least one lens having a cylinder effect. For example, the optical system can comprise a magazine containing a number of spherical lenses and/or cylinder lenses, which each have different spherical or cylinder effects, and wherein the magazine is configured and arranged such that individual spherical lenses or individual cylinder lens and/or a combination of a plurality of spherical lenses or cylinder lenses from the magazine can be selected and used to project the target. The optical system can e.g. also comprise an Alvarez lens system. In other words, a target (or a projected or virtual target) to the test subject through which the test subject sees the target or virtual target. The optical system can e.g. also comprise two lenses that are rotatable relative to one another and each have at least one cylinder component in the effects. In particular, the optical system can comprise two cylinder lenses comprising rotationally symmetrical surfaces, preferably planar surfaces, which face one another and interlock. The optical system can also comprise a positive cylinder lens and a negative cylinder lens having an identical but opposite effect, which are mounted to be rotatable relative to one another and are preferably movable relative to one another.

Furthermore, it may be that the viewing angle of the target changes while applying different effects by means of the optical system. This can either be prevented by an appropriate construction of the optical system or can be determined by a computer and compensated for when displayed. For this purpose, the viewing angle needs to be determined on the basis of the applied effect and a visual acuity value needs to be assigned on the basis of this actual viewing angle, which can be achieved e.g. by determining the magnification of the optical system and displaying the target in an accordingly scaled-down manner. Alternatively, the optical system can be calibrated by means of a camera in that the size of the target can be implemented directly by a camera arranged at the point of the at least one eye of the test subject (and looking into the optical system).

The feedback from the test subject or the test subject action can be verbal in principle. In this case, a user can note the state of the optical system at the time of the feedback or test subject action and/or relay the feedback directly to the feedback system. This variant is, however, prone to errors and causes delays. This is why direct feedback from the test subject to the feedback system is preferred. For this purpose, the feedback system can comprise a button in the simplest case. In another preferred embodiment, the feedback system can also comprise two buttons (“+” and “−”), three buttons (“+”, “−” and “OK”), four buttons (e.g. “+”, “−”, “OK” and “Cancel”), etc., and/or can comprise a joystick. Alternatively, or additionally, the feedback system can comprise a microphone for capturing verbal comments from the test subject.

In a preferred embodiment, the device comprises an evaluation unit for determining the sensitivity of the at least one eye of the test subject based on at least two provided pairs of values for visual acuity and refraction. In this case, the visual acuity limiting refraction determination unit can be a component of the evaluation unit. In other words, the evaluation unit can comprise the visual acuity limiting refraction determination unit.

In another preferred embodiment, the device comprises an auto-refractometric or aberrometric measuring unit for determining one or more objective refractions of the at least one eye of the test subject, wherein the auto-refractometric or aberrometric measuring unit is preferably configured to measure and/or monitor an accommodation state of the at least one eye of the test subject. Furthermore, the auto-refractometric or aberrometric measuring unit can comprise a camera for determining a pupil size (in particular a pupil radius) of the at least one eye of the test subject. Alternatively, or additionally, the auto-refractometric or aberrometric measuring unit can comprise a calibration camera for calibrating the optical system. The camera for determining a pupil size and the calibration camera can also be implemented in one single camera which combines both functions (determining the pupil size and calibrating the optical system).

In another preferred embodiment, the device comprises a pupil size measuring unit (in particular a camera) for determining a pupil size (in particular a pupil radius) of the at least one eye of the test subject. Alternatively, or additionally, the device can comprise a lighting device for generating at least two brightness levels. Alternatively, or additionally, the device can comprise a pupillometer device which is configured to detect first pupillometric data of the at least one eye at a first brightness and to detect secondary pupillometric data of the at least one eye at a second brightness.

A further aspect for solving the problem relates to a device for calculating, optimizing or evaluating a spectacle lens for at least one eye of a test subject taking into account the sensitivity of the at least one eye of the test subject, comprising a device according to the invention for determining the sensitivity of the at least one eye of the spectacle wearer.

The device for calculating, optimizing or evaluating a spectacle lens can in particular comprise the following components:
a surface model database for specifying a first surface and a second surface for the spectacle lens to be calculated or optimized;
a main beam determining module for determining the course of a main beam through at least one vision point of at least one surface of the spectacle lens to be calculated or optimized into a model eye;
an evaluation module for evaluating an aberration of a wavefront resulting along the main beam from a spherical wavefront impinging on the first surface of the spectacle lens at an evaluation surface in comparison with a wavefront converging in one point on the retina of the eye model; and
an optimization module for iteratively varying the at least one surface of the spectacle lens to be calculated or optimized until the evaluated aberration corresponds to a predetermined target aberration.

Another aspect for solving the problem relates to a device for manufacturing a spectacle lens, comprising:
calculation or optimization means which are configured to calculate or optimize the spectacle lens according to a method according to the invention for calculating or optimizing a spectacle lens; and
processing means which are configured to process the spectacle lens according to the result of the calculation or optimization.

Another aspect for solving the problem relates to a spectacle lens which has been produced by means of a method according to the invention for manufacturing a spectacle lens and/or by means of a device according to the invention for manufacturing a spectacle lens.

In addition, the invention provides the use of a spectacle lens manufactured in accordance with the manufacturing method according to the present invention, in particular in a preferred embodiment, in a predetermined average or customized position of use of the spectacle lens in front of the eye of a particular spectacle wearer for correcting an ametropia of the spectacle wearer.

In particular, a computer-implemented method according to the invention can be provided in the form of ordering software and/or industry software. In particular, the data required for calculating and/or optimizing and/or manufacturing a spectacle lens can be acquired and/or transmitted in a method of this kind.

A device according to the invention and/or a system according to the invention, e.g. for ordering a spectacle lens, can in particular comprise a computer and/or a data server, which is configured to communicate over a network (e.g. Internet). The computer is in particular configured to execute a computer-implemented method, e.g. ordering software for ordering at least one spectacle lens, and/or transfer software for transferring relevant data, and/or determination software for determining relevant data, and/or calculation or optimization software for calculating and/or optimizing a spectacle lens to be manufactured, according to the present invention.

It goes without saying that the features described above and hereinafter can be used not only in the combination stated, but also in isolation or in other combinations, without departing from the scope of the present invention.

The comments made above or below with regard to the embodiments of the first aspect are also applicable to the further independent aspects described above and in particular to preferred embodiments in this regard. In particular, the comments made above or below with regard to the embodiments of the other independent aspects are also applicable to one independent aspect of the present invention and to preferred embodiments in this regard.

Individual embodiments for solving the problem are described in the following by way of example with reference to the drawings. In this case, the individual embodiments described sometimes contain features that are not absolutely necessary for reproducing the claimed subject matter but which provide desired properties in certain applications. Therefore, embodiments which do not contain all the features of the embodiments described in the following should also be considered to be disclosed as falling under the described technical teaching. Furthermore, in order to avoid any unnecessary repetition, certain features are only mentioned in relation to individual embodiments described in the following. It should be noted that the individual embodiments therefore should not only be considered in isolation but should also be considered together. When considering these embodiments together, a person skilled in the art will note that individual embodiments can also be modified by including individual or multiple features of other embodiments. It should be noted that systematically combining the individual embodiments with individual or multiple features that are described in relation to other embodiments can be desirable and useful, and therefore should be taken into consideration and also considered as being covered by the description.

### BRIEF DESCRIPTION OF THE DRAWINGS

FIG. 1 shows an exemplary image or photograph which gives the observer a sense of distance;

FIG. 2 shows the image or photograph from FIG. 1 with exemplary optotypes integrated in the image or overlaid on the image;

FIG. 3 shows a graph of the amplitude of accommodation as a function of age (Duane's curve).

### DETAILED DESCRIPTION

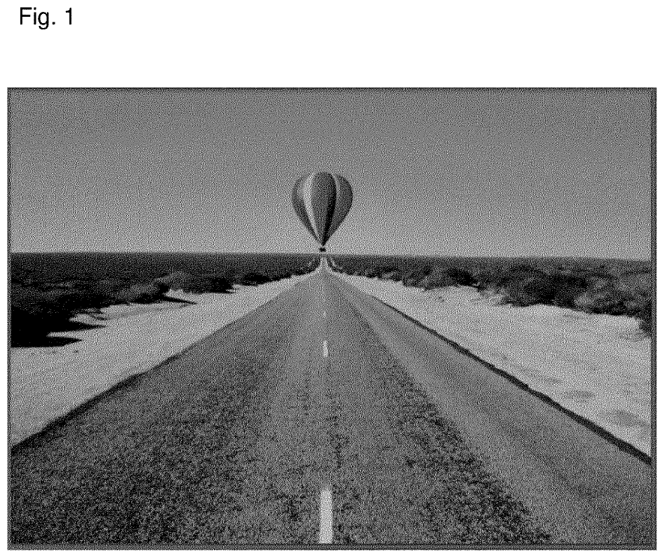

FIG. 1 shows an exemplary image or photograph which contains a hot-air balloon and a road and gives the observer a sense of distance. In the context of the present invention, an image can e.g. be projected as a target (in particular as a virtual target) into the at least one eye of a test subject in order to, for example, carry out an objective refraction measurement in a fogged state in which the test subject only recognizes the image or details of the image out of focus.

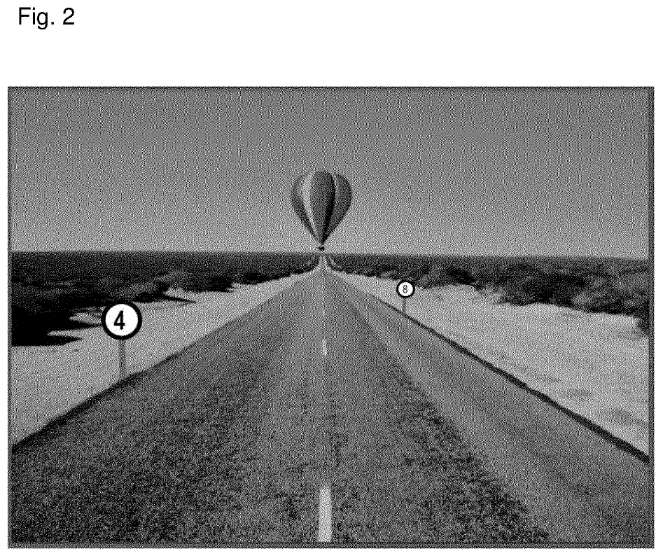

FIG. 2 shows the image or photograph from FIG. 1 with exemplary optotypes integrated in the image or overlaid on the image, namely numbers of different sizes arranged in road signs. Each of these optotypes has a predetermined visual acuity or a predetermined visual acuity level. In the context of the method according to the invention, the image is provided with the optotype by means of an optical system at an adjustable target refraction. This target refraction is varied by means of the optical system and the test subject signals by means of a test subject action that the identifiability of the target or optotype for them has changed at the time of the test subject action. In this way, pairs of values for visual acuity and refraction can be provided in order to determine the sensitivity of the at least one eye of the test subject.

One or more targets can be provided to the test subject or can be projected as virtual targets into the at least one eye of the test subject. Depending on the embodiment, two or more targets can be used, which can also be identical in content.

For instance, a first target can e.g. be an image that gives a sense of distance (see e.g. FIG. 1), a second target can be one or more optotypes of a certain size, and a third target can be one or more optotypes of a different size. Here, optotypes are understood to be all the symbols, images and the like that can be identified by a test subject.

Alternatively, the first target can be an image that gives a sense of distance while the second and third target can be identical in content and contain one or more optotypes, each in one of two sizes.

Alternatively, all three targets can be identical and represent one image that gives a sense of distance, but can contain one or more details, the recognition of each of which can be assigned to a visual acuity level. These details are expressly included in this description of the term “optotype”. Examples of details of this kind are in an image that e.g. contains a hot-air balloon and a road:
Symbols or panels on a hot-air balloon and the basket of a hot-air balloon,
clouds or symbols in clouds,
lines on a road, and/or
symbols on signs at the side of the road.

A particularly suitable symbol is e.g. one or more concentric rings, which merge together to form a circle at a given lack of focus.

In the present invention, by contrast with the prior art, for determining the sensitivity, the visual acuity level for a certain applied effect is not determined, but the applied effect necessary for reaching a predetermined visual acuity is determined. Furthermore, the determination of the visual acuity can be linked to the measurement of auto-refractometric or aberrometric data in the non-accommodated or accommodated state. In a specific embodiment, the accommodation state of the eye can also be tracked, in order to thus obtain even more reliable values for the sensitivity.

### A. Approach According to an Exemplary Embodiment Without Subjective Refraction

A test on the test subject can be carried out as follows, for example:
1) The objective refraction value of the test subject is determined by means of an auto-refractometric or aberrometric measurement. To do this, a first target is presented to the test subject. In this case, a suitable optical system is used to provide a first effect to the test subject which does not allow them to recognize the target fully in focus, in order to therefore make the ciliary muscles relax.
2) A second target is then provided to the test subject and a second effect is applied by means of the test optical system in which a test subject cannot recognize the optotype(s) with high visual acuity. This is in particular achieved by a spherical effect being selected which corresponds to the central sphere or to one of the two main sections of the objective refraction value plus an additional positive spherical effect. The latter effect, often called “fogging”, is therefore selected, since the test subject cannot compensate for this kind of effect by accommodation. To establish the speed at which the provided effect is changed, standard values on the basis of means for a number of test subjects can be used. For example, it is known that, with fogging, the visual acuity is approximately halved by 0.5 dpt sphere and 1 dpt cylinder. Preferably, the target refraction is varied at a speed of between 1/16 dpt per second and ½ dpt per second. The additional spherical effect can also be dependent on the pupil measured by the aberrometry unit. It can e.g. be reciprocally proportional to the pupil radius, such that test subjects having smaller pupils are preferably fogged with a stronger effect than test subjects having larger pupils, in order to ensure that the lack of focus perceived by all the test subjects is similar.
3) Alternatively, a sphero-cylindrical effect can also be provided. For example, a cylindrical effect can be adopted for the optical system from the objective refraction and an additional positive spherical effect can be applied to a mean objective refraction value. Alternatively, or additionally, an astigmatic offset can be applied to the objective refraction value (so-called astigmatic fogging). The optical effect is then slowly (e.g. between 1/16 dpt per second and ½ dpt per second) changed towards optimum or objective refraction (by varying the spherical effect and/or varying the astigmatic effect).
4) Once the test subject can recognize the optotype of the second target by the effect being changed, they communicate this (e.g. using the “OK” button). Optionally, they can adjust the limiting effect themselves (e.g. using the “+” and “−” buttons) and activate it (e.g. likewise using the “OK” button). The effect adjusted here is stored as the “visual acuity limiting effect” or “visual acuity limiting refraction” when recognizing the second target.
5) The third target is provided to the test subject.
6) Again, the optical effect is then slowly (e.g. between 1/16 dpt per second and ½ dpt per second) changed towards the optimum or objective refraction value (by varying the spherical and/or astigmatic effect).
7) Once the test subject can recognize the optotype of the third target by the effect being changed, they communicate this (e.g. using the “OK” button). Optionally, they can adjust the limiting effect (e.g. using the “+” and “−” buttons) and activate it (e.g. likewise using the “OK” button). The effect adjusted here is stored as the “visual acuity limiting effect” or “visual acuity limiting refraction” when recognizing the third target.

The sensitivity can be determined from the visual acuity levels of the two targets, the objective refraction value, the effect when recognizing the second target and the effect when recognizing the third target. For this purpose, a sensitivity metric, as has been described above in exemplary embodiments, can be used in particular. Here, the false refractions result from the spherical and/or astigmatic distance of the effect when recognizing the relevant target from the objective refraction value.

### B. Approach According to an Exemplary Embodiment with Subjective Refraction

In this variant, the above-mentioned steps 5) to 7) can be omitted from the approach under section A. Therefore, only the visual acuity for one target and the effect when recognizing a target need to be determined. A subjective refraction determination is then carried out and, in the process, the subjective refraction value and the visual acuity (visual acuity cum correctione (VAcc)) that the test subject achieves thereby is determined. In this process, the objective refraction value can be used as the starting value for the subjective refraction determination.

Alternatively, the subjective refraction determination with visual acuity determination can be carried out before the steps from section A. In this case, the auto-refractometry or aberrometry do not need to be carried out and the objective refraction value (step 1) does not need to be determined, and the subjective refraction value is used at this point.

Here, the false refraction can be as the spherical or astigmatic distance of the effect when recognizing the target from the subjective refraction value.

Instead of the subjective refraction value, a combined refraction value can also be used to calculate the sensitivity or false refraction. This can be calculated on the basis of the subjective refraction value and the objective refraction value or other data (e.g. lower-order or higher-order aberrations from the aberrometry or other biometric data such as the shape of the cornea, lens-to-retina distance, anterior chamber depth).

### C. Adjusting the Visual Acuity Level of a Target

Furthermore, the at least one visual acuity level of the symbol or symbols of a target can be adjusted to the test subject. This is useful, for example, if an astigmatism of the test subject cannot be compensated for. The visual acuity level of the (virtual) target can then be selected such that the target can still be recognized despite the false refraction remaining owing to the astigmatism.

Information regarding the eyesight (e.g. visual acuity cum correctione or visual acuity sine correctione, for example from the subjective refraction determination) can feed into the determination of the target size.

If the test subject cannot recognize the symbol despite a low deviation of the applied effect from the objective, subjective or combined refraction value, a switch can be made to a lower visual acuity level and the corresponding step can be repeated with a lower visual acuity level.

Additionally, or alternatively, the findings from step 4 can be included in the determination of the visual acuity level in step 6.

In order to avoid the test subject already having recognized the optotype in multiple measurements or when switching between eyes, the at least one optotype or symbol or detail in the image can be changed between different measurements or when switching between eyes (e.g. rotation of a Landolt ring or changing a letter or number). For this purpose, electronic displays are of course particularly suitable as the target providing device.

### D. Finding the Out-of-Focus Point and Adjustment of the Effect by the Test Subject

### Finding the Out-of-Focus Point

As an alternative to the approach in the above sections, the effect applied at the start (i.e. in step 2) according to section A or B can also be an effect that allows the target to be recognized. This can be an objective, subjective or combined refraction value.

In steps 5) and 6), the applied effect is then removed from this effect in the plus direction. This direction is selected in order to avoid accommodation. In steps 4) and 7), the test subject then signals the time at which they can no longer recognize the optotype.

If, analogously to the approach in section A, the applied effects are determined for two visual acuity levels, in this case the applied effects can first (steps 2-4) be determined for the higher visual acuity level and then (steps 5-7) for the lower visual acuity level. In this way, the false refraction can be increased over the course of the approach, meaning that the optotype first becomes unrecognizable with the more difficult recognizability (higher visual acuity level) and then with the easier recognizability (lower visual acuity level).

Correcting the (Out-of-Focus) in-Focus Point

In steps 4) and 7) of the above embodiments, the test subject can optionally correct the applied effect if they are not sure that they have signalled the correct time or the correct applied effect. This can for example be done using the “+” and “−” buttons on the feedback unit.

Adjustment of the (Out-of-Focus) in-Focus Point by the Test Subject

The test subject can also be directly asked to adjust the applied effect at which it is still possible or no longer possible for them to recognize the optotype themselves. This can for example be done using the “+” and “−” buttons on the feedback unit.

### Approaching the (Out-of-Focus) in-Focus Point from Different Directions

Furthermore, one out-of-focus point can be determined when increasing and another out-of-focus point can be determined when decreasing. These points can be different from one another and can be subsequently averaged. Alternatively, the sensitivity can be determined from both out-of-focus points as part of a method of least squares by means of known metrics.

### Repeating the Measurement

Of course, the out-of-focus points can also be determined multiple times in order to increase the measurement accuracy of the method.

### Monitoring the Accommodation State

During steps 3), 4), 6) and 7) in the method according to section A or during step 3) or 4) in the method according to section B, the accommodation state of the at least one eye of the test subject can be monitored by means of the auto-refractometry or aberrometry unit. The results obtained therefrom can be used for controlling the progress (e.g. terminating or repeating individual steps in the event of undesired accommodation (e.g. values falling below a certain threshold)). The measurement can be carried out both continuously and only when signalling the recognizability. Furthermore, a measured accommodation state (sphere, cylinder, lower-order or higher-order aberrations), ideally when signalling the recognizability, can be included in the calculation of the sensitivity or the false refraction.

### E. Lack of Focus in the Minus Direction and Incorporating a Near-Vision Measurement

### Lack of Focus in the Minus Direction

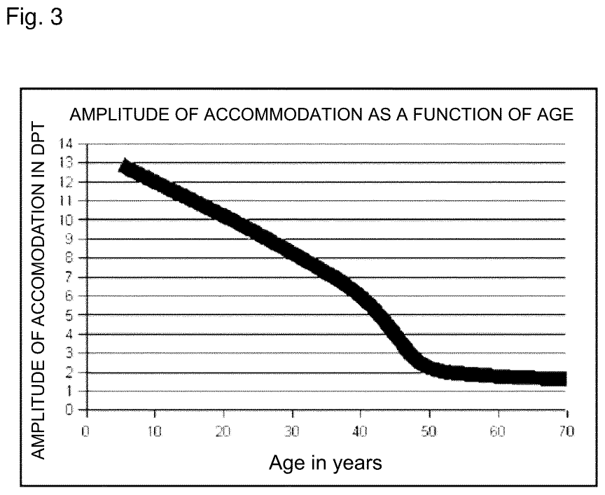

In the above exemplary embodiments, the applied effect corresponds to a false refraction in the plus direction since this cannot be compensated for by the test subject by accommodation. The reverse can also be implemented, i.e. with an applied effect that corresponds to a false refraction in the minus direction. The accommodation that potentially occurs in that case can be dealt with as follows:
ignoring the accommodation;
measuring test subjects who are e.g. physiologically (e.g. in an age-related manner) or pharmacologically (e.g. by drops) affected or can only accommodate to a poor extent;
measuring or monitoring the accommodation state;
using assumptions on the accommodation ability (e.g. on the basis of age according to Duane's curve; see FIG. 3).

The curve shown in FIG. 3 by Duane is taken from B. Lachenmayr, D. Friedburg, E. Hartmann, A. Buser: “Auge-Brille-Refraktion: Schober-Kurs: verstehen-lernen-anwenden” [Eye-Spectacles-Refraction: Schober course: understand-learn-apply], 2005, figure 1.29, and was originally published in Alexander Duane: “Studies in monocular and binocular accommodation with their clinical applications”, Transactions of the American Ophthalmological Society, volume 20, 1922, pages 132-157, PMID 16692582, PMC 1318318. Duane's curve shows that the human eye's ability to accommodate (amplitude of accommodation) continuously decreases from the age of eight to just after the age of 50 on average from 14 to one diopter.

The influence of accommodation on the sphere can be taken into account here in the following ways, for example:
The accommodation value is subtracted from the value of the distance of the applied effect from the refraction value for distance vision;
The false refraction is calculated directly from the applied effect and the measured or assumed refraction value.

In an analogous manner, the astigmatic deviation can be calculated in accordance with the known formalisms (e.g. cross cylinder formula, power vector notation) by way of the measured cylinder in order to take into account a change in the astigmatism owing to the accommodation. Furthermore, measured higher-order aberrations can be taken into account by way of known metrics.

### Incorporating a Near-Vision Measurement

The above-described approach can be combined with a determination of the objective near-vision refraction values, the maximum accommodation and/or the (lower-order or higher-order) aberrations.

For this purpose, the process can be as follows: the accommodation state of the eye is monitored by means of auto-refractometric or aberrometric measurements (which are ideally concurrent and as frequent as possible). An applied effect that allows the target to be recognized is used as a starting point. This can be an objective, subjective or combined refraction value. In step 5) and optionally in step 6), the applied effect is then removed from this effect in the plus direction. In step 4) and optionally in step 7), the test subject then signals the time at which they can no longer recognize the optotype. If the applied effects are determined for two visual acuity levels, in this case the applied effects can first (steps 2-4) be determined for the higher visual acuity level and then (steps 5-7) for the lower visual acuity level. In this way, the false refraction can be increased over the course of the approach, meaning that the optotype first becomes unrecognizable with the more difficult recognizability (higher visual acuity level) and then with the easier recognizability (lower visual acuity level). The auto-refractometric or aberrometric value measured when signalling the loss of the recognizability (each time) is used for calculating the sensitivity or visual acuity.

The value of the auto-refractometric or aberrometric measurement that corresponds to the greatest accommodation is then used as the value (sphere, cylinder, lower-order or higher-order aberrations) for the near-vision refraction or for the maximum accommodation ability.

### F. Monitoring the Pupil Size

Furthermore, the pupil size (e.g. in the form of the pupil radius) can be monitored, e.g. by means of a camera arranged in an auto-refractometer or aberrometer, or by means of a separate camera. The pupil size measured at the out-of-focus point or accordingly just before (e.g. up to 2 seconds before the out-of-focus point is reached) can be used when determining the sensitivity to a lack of focus.

The measured pupil size can then be used to quantify the lack of focus of the image on the retina by means of a suitably parameterized eye model and the known additional fogging. For instance, the angle can be calculated at which the disk of confusion of an out-of-focus point can be observed in a given pupil and with given additional fogging (cf. WO 2019 034525 A1). The sensitivity can be determined as part of a visual acuity model of this kind as a deterioration in the sharpness of vision per angle of the disk of confusion.

### G. More Complex Models for the Sensitivity

In more complex models, a distinction can be made between the influence of spherical fogging or false refractions and the astigmatic fogging or false refractions. For this purpose, spherical fogging and astigmatic fogging can be determined for the same visual acuity level.

### I. Combination with Other Measurements

The present invention can be very effectively combined with or embedded in other measurements. In a preferred embodiment, the approach according to section A or B is carried out after an auto-refractometric or aberrometric measurement for distance vision. In this case, this auto-refractometric or aberrometric distance-vision measurement already constitutes the first step according to section A and does not need to be carried out again. In this case, the approach according to one of the above sections can be carried out either before or after any potential measurement. The first approach has the advantage that the (virtual) target is still not known to the test subject at first and the test subject has already become familiar with the target for the near-vision measurement.

1. A method for determining a sensitivity of at least one eye of a test subject based on at least two provided pairs of values for visual acuity and refraction, wherein at least one of the pairs of values for visual acuity and refraction is provided by:
projecting a target with an adjustable target refraction into the at least one eye of the test subject, wherein the target is configured to verify a predetermined visual acuity; and
determining a visual acuity limiting refraction of the at least one eye of the test subject associated with the predetermined visual acuity by varying the target refraction of the target projected into the at least one eye of the test subject and detecting a test subject action which results in a determination that an identifiability of the target for the test subject has changed at a time of the test subject action,
wherein the sensitivity is determined based on at least one calculated false refraction, wherein the at least one calculated false refraction is calculated based on the determined optimum refraction.

2. The method according to claim 1,
wherein prior to the step of projecting a target, which is configured to verify a predetermined visual acuity, into the at least one eye of the test subject, an objective and/or subjective refraction result of the at least one eye of the test subject is determined, and
wherein, preferably prior to the step of varying the target refraction, the target is projected into the at least one eye of the test subject with such a starting target refraction that the test subject can only recognize the target out of focus and/or cannot identify it.

3. The method according to claim 1, further comprising the step of
determining an optimum refraction of the at least one eye of the test subject and determining the visual acuity achieved by the at least one eye of the test subject when compensating for a possible ametropia of the at least one eye of the test subject based on the determined optimum refraction.

4. The method according to claim 1, further comprising the steps of:
determining a subjective refraction result for at least one eye of the test subject; and
determining the visual acuity achieved by the at least one eye of the test subject when compensating for a possible ametropia of the at least one eye of the test subject based on the determined subjective refraction result,
wherein the optimal refraction of the at least one eye of the test subject is determined based on the subjective refraction result and an objective refraction result.

5. (canceled)

6. The method according to claim 1, wherein varying the target refraction comprises monotonically decreasing the target refraction and/or monotonically increasing the target refraction.

7. The method according to claim 1, wherein the determination of a visual acuity limiting refraction of the at least one eye of the test subject associated with the predetermined visual acuity is carried out by decreasing the target refraction and detecting a test subject action while decreasing of the target refraction, and/or by increasing the target refraction and detecting a test subject action while increasing of the target refraction, wherein each test subject action results in a determination that the identifiability of the target for the test subject has changed at the time of the respective test subject action.

8. The method according to claim 1, wherein at least two of the provided pairs of values for visual acuity and refraction are provided by:
projecting a first target with a first adjustable target refraction into the at least one eye of the test subject, wherein the first target is configured to verify a predetermined first visual acuity;
determining a first visual acuity limiting refraction of the at least one eye of the test subject associated with the predetermined first visual acuity by varying the first target refraction of the first target projected into the at least one eye of the test subject and detecting a first test subject action which results in a determination that the identifiability of the first target for the test subject has changed at the time of the first test subject action;
projecting a second target with a second adjustable target refraction into the at least one eye of the test subject, wherein the second target is configured to verify a predetermined second visual acuity which differs from the predetermined first visual acuity; and
determining a second visual acuity limiting refraction of the at least one eye of the test subject associated with the predetermined second visual acuity by varying the second target refraction of the second target projected into the at least one eye of the test subject and detecting a second test subject action which results in a determination that the identifiability of the second target for the test subject has changed at the time of the second test subject action.

9. The method according to claim 1, wherein determining a visual acuity limiting refraction comprises measuring an accommodation state and/or a pupil size of the at least one eye of the test subject, wherein measuring the accommodation state and/or the pupil size is performed in particular at the time of or immediately after the test subject action.

10. The method according to claim 1, wherein the test subject is presented with a visual task with at least two possible different answers in order to determine a visual acuity limiting refraction, and wherein the test subject can answer the visual task using the test subject action.

11. The method according to claim 1,
wherein, prior to the step of determining a visual acuity limiting refraction, first aberrometric data of the at least one eye of the test subject, in particular for a distance accommodation state of the at least one eye of the test subject, are acquired, and
wherein preferably the method further comprises acquiring second aberrometric data of the at least one eye of the test subject for a near accommodation state of the at least one eye of the test subject, wherein the acquiring of second aberrometric data preferably occurs prior to the step of determining a visual acuity limiting refraction.

12. A method for calculating, optimizing or evaluating a spectacle lens for at least one eye of a test subject taking into account the sensitivity of the at least one eye of the test subject, wherein the sensitivity of the at least one eye of the test subject is determined by a method according to claim 1.

13. A method of manufacturing a spectacle lens, comprising:
calculating or optimizing a spectacle lens according to the method of calculating or optimizing a spectacle lens according to claim 12; and
manufacturing the spectacle lens calculated or optimized in this way.

14. A non-transitory computer program product, comprising program code which is adapted and configured, when loaded and executed on a computer, to perform a method according to claim 1.

15. A device for determining a sensitivity of at least one eye of a test subject, comprising:
a target providing device configured to provide a target which is configured to verify a predetermined visual acuity;
an optical system configured to project the target with a target refraction into the at least one eye of the test subject, wherein the optical system is configured to adjust and vary the target refraction;
a feedback unit configured to detect a test subject action in order to determine that an identifiability of the target for the test subject has changed at a time of the test subject action, in particular as a result of varying the target refraction of the target projected into the at least one eye of the test subject with aid of the optical system; and
a visual acuity limiting refraction determination unit configured to detect a visual acuity limiting refraction of the at least one eye of the test subject associated with the predetermined visual acuity, wherein the visual acuity limiting refraction determination unit is configured to detect the target refraction caused by the optical system at the time of the test subject action, and
an evaluation unit configured to determine the sensitivity based on at least one calculated false refraction that is calculated based on a determined optimum refraction of the at least one eye of the test subject.

16. The device according to claim 15, wherein the evaluation unit is configured to determine the sensitivity of the at least one eye of the test subject based on at least two provided pairs of values for visual acuity and refraction, and wherein the visual acuity limiting refraction determination unit is a component of the evaluation unit.

17. The device according to claim 15, further comprising:
an auto-refractometric or aberrometric measuring unit configured to determine one or more objective refractions of the at least one eye of the test subject, wherein the auto-refractometric or aberrometric measuring unit is configured to measure and/or monitor an accommodation state of the at least one eye of the test subject.

18. The device according to claim 15, further comprising:
a pupil size measuring unit configured to determine a pupil size of the at least one eye of the test subject; and/or
a lighting device configured to generate at least two brightness levels, and/or
a pupillometer device which is configured to detect first pupillometric data of the at least one eye at a first brightness and to detect secondary pupillometric data of the at least one eye at a second brightness.

19. A system for calculating, optimizing or evaluating a spectacle lens for at least one eye of a test subject taking into account the sensitivity of the at least one eye of the test subject, comprising a device for determining the sensitivity of the at least one eye of a spectacle wearer according to claim 15.

20. A device for manufacturing a spectacle lens, comprising:
a calculator or optimizer which are configured to calculate or optimize the spectacle lens according to a method for calculating or optimizing a spectacle lens according to claim 12; and
processors which are configured to process the spectacle lens according to the result of the calculation or optimization.

21. A spectacle lens, which has been produced using a method according to claim 13.
